(19) United States
(12) Patent Application Publication
(10) Pub. No.: US 2020/0093986 A1
(43) Pub. Date: Mar. 26, 2020

(54) KIT FOR THE MODULAR CONFIGURATION OF A MEDICAL PUMP DEVICE, AND MEDICAL PUMP DEVICE

(71) Applicant: B. BRAUN MELSUNGEN AG, Melsungen (DE)

(72) Inventors: Karsten HASLBECK, Melsungen (DE); Alexander David KENT, Korle (DE); Jens STETTNER, Melsungen (DE)

(21) Appl. No.: 16/569,981

(22) Filed: Sep. 13, 2019

(30) Foreign Application Priority Data
Sep. 26, 2018 (DE) 10 2018 216 508.4

(51) Int. Cl. A61M 5/168; A61M 5/142

(52) U.S. Cl. CPC A61M 5/16804; A61M 5/172; A61M 5/16877; A61M 5/142

(57) ABSTRACT

A kit is used for the modular configuration of a medical pump device for delivering a medical fluid. Such a medical pump device can be used in infusion therapy.

### CROSS REFERENCE TO RELATED APPLICATION(S)

This application claims the benefit of priority of German Application No. 10 2018 216 508.4, filed Sep. 26, 2018, the content of which is incorporated by reference herein in its entirety.

### FIELD

The present disclosure relates to a kit for the modular configuration of a medical pump device for delivering a medical fluid, and to such a medical pump device.

### BACKGROUND

Medical pump devices are generally known in the field of medical technology and are provided for use in infusion therapy. The known medical pump device has a pump unit, which can also be referred to as an elastomer infusion pump or medical elastomer pump. The elastomer pump has an elastomer membrane, which forms a pump volume for receiving and delivering a medical fluid. During filling of the pump volume with the medical fluid, the elastomer membrane is extended elastically like a balloon. The membrane extended in this way produces a delivery pressure on the pump volume. By means of the delivery pressure, the medical fluid can be delivered, for example, into a catheter line, connectable in a fluid-conducting manner to the pump device, and can thus be administered to a patient. The known medical pump device moreover has a throttle unit connected in a fluid-conducting manner to the pump volume. The throttle unit is designed to throttle the delivery of the medical fluid to a nominal flow rate and for this purpose has a throttle effect in terms of flow mechanics. This throttle effect is not changeable. In the known medical pump device, the pump unit and the throttle unit are joined non-releasably to each other at the production site. As a result, the known medical pump device has a fixed specification in terms of a maximum fluid quantity that can be administered and in terms of a nominal flow rate during the administration of the medical fluid. Different medical cases of use require different maximum fluid quantities that can be administered, and thus differently dimensioned pump volumes, and also different nominal flow rates, and thus differently dimensioned throttle effects. This means that the known medical pump device has to be made available in a wide variety of specifications, i.e. with a large specification range. This in particular makes production and storage difficult.

### SUMMARY

The object of the present disclosure is to permit the design of a medical pump device of the type mentioned at the outset, wherein simplified production and simplified storage are in particular intended to be permitted while at the same time maintaining or extending the specification range.

This object is achieved by a kit.

The kit according to the present disclosure is provided for the modular configuration of a medical pump device for delivering a medical fluid and has
a group with a plurality of different pump units, which each have a pump volume for receiving and delivering the medical fluid,
wherein the pump units have differently dimensioned pump volumes,
and wherein the pump units each have a first coupling element, which is designed in such a way that the respective pump volume is connectable to a throttle unit in a fluid-conducting manner,
and a group with a plurality of different throttle units, which are each designed to throttle the delivery of the medical fluid,
wherein the throttle units have differently dimensioned throttle effects,
and wherein the throttle units each have a second coupling element, which is designed in such a way that the respective throttle unit is connectable in a fluid-conducting manner to a pump volume of one of the pump units,
wherein the pump units and the throttle units are arranged spatially separated from each other.

By means of the solution according to the present disclosure, and depending on the medical case of use, it is possible to configure a medical pump device having a pump volume required for this case of use and having a flow rate required for this case of use. This configuration of the medical pump device using the kit according to the present disclosure is carried out individually by the end user and to this extent by a patient or by medical personnel. For this purpose, the person in question chooses, from the group of different pump units, a pump unit having the required pump volume, and, from the group of throttle units, a throttle unit having the required throttle effect. Thereafter, the selected pump unit and the selected throttle unit are connected to each other in a fluid-conducting manner, and preferably non-releasably, by means of the coupling elements. In this way, the solution according to the present disclosure permits much simpler production and storage while at the same time maintaining or extending the specification range. The reason for this is that it is possible to dispense with producing and storing medical pump devices having very different specifications in terms of a fixed combination of pump volume and throttle effect. Moreover the solution according to the present disclosure permits suitably simplified logistics, since a reduced number of articles and/or a reduced transport volume has to be transported. A kit according to the present disclosure with for example three different pump units and three different throttle units has six parts and permits a modular configuration of a medical pump device in nine different specifications. Only six parts, i.e. three pump units and three throttle units, need to be produced, transported and stored, instead of nine differently specified medical pump devices. A kit according to the present disclosure with for example four different pump units and four different throttle units has eight parts and permits a modular configuration of a medical pump device in 16 different specifications. Only eight parts, i.e. four pump units and four throttle units, need to be produced, transported and stored, instead of 16 differently specified medical pump devices. A kit according to the present disclosure with for example ten different pump units and ten different throttle units has 20 parts and permits a modular configuration of a medical pump device in 100 different specifications. Only 20 parts, i.e. 10 pump units and 10 throttle units, need to be produced, transported and stored, instead of 100 differently specified medical pump devices. The pump units are preferably designed in such a way that a supply with external and in particular electrical operating energy for delivering the medical fluid is not needed. The pump units are preferably each designed in the manner of an elastomer pump. Elastomer pumps are in principle known as such in the field of medical technology. Alternatively, the pump units can be supplied with operating energy via a gas pressure or spring energy reservoir. Further alternatively, the pump units can be configured as electric and/or electronic pump units, wherein supplying with electrical operating energy is provided. In such a configuration, the pump unit can actively control a delivery volume. The group of different pump units preferably has a large number of different pump units. Here, different means being of the same design and being differently dimensioned as regards a capacity of the pump volume. Their pump volumes are preferably dimensioned to a nominal capacity of between 25 ml and 1000 ml. For example, the pump volumes can be differently dimensioned to nominally 50 ml, 60 ml, 100 ml, 120 ml, 125 ml, 250 ml, 270 ml, 400 ml and/or 500 ml. The throttle units each preferably act, in terms of flow mechanics, in the manner of a flow throttle. Flow throttles are in principle known as such in flow technology. The group of different throttle units preferably has a large number of different throttle units. Here, different means being of the same design and being differently dimensioned as regards the throttle effect. The throttle effect can, for example, be differently dimensioned in such a way that the delivery of the medical fluid takes place with a nominal flow rate of between 0.5 ml/h and 1,000 ml/h. For example, the throttle effects can be differently dimensioned to flow rates of 2 ml/h, 4 ml/h, 5 ml/h, 10 ml/h, 50 ml/h, 100 ml/h, 175 ml/h, 200 ml/h, 250 ml/h and/or 500 ml/h. Alternatively or in addition, the throttle effects of the different throttle units can be different in view of tolerance and/or accuracy of the respectively achievable flow rate. Put in other words, the throttle units can have tolerances and/or accuracies of the throttle effect in different grading. The throttle effect of the throttle units is preferably unchangeable in the sense of not being adjustable and/or not being controllable by the end user. Alternatively, the throttle effect can be adjustable by means of a corresponding control device. Further alternatively, the throttle units can be configured as electrically and/or electronically controllable and/or regulatable throttle units. For example, such a throttle unit can be a piezo element which is transferred to a dedicated regulating position by a capacitance, or be configured as an electronically controllable valve. The first coupling element and the second coupling element are designed complementing each other. In a state when connected to each other, the coupling elements form a fluid-conducting connection between the pump volume and the throttle unit. The coupling elements can in particular be designed as plug and/or screw connectors. The kit according to the present disclosure provides a spatially separated arrangement of the pump units and of the throttle units when they are not connected to each other in a fluid-conducting manner. Instead, the fluid-conducting connection is produced only by the end user, for example by a patient or by medical personnel, in a modular configuration of the medical pump device using the kit. Preferably, the pump units and the throttle units can include mutually complementary electrical and/or electro-mechanical interfaces, wherein in the connected state of the interfaces information can be exchanged. Such information can involve, for example, an identification number, a specification, or the like. Moreover, during contacting of the interfaces, there can be a kind of invalidation occurring, if the pump unit and/or the throttle unit are disposable products provided for one-time use.

In one embodiment of the present disclosure, the pump units each have an elastomer membrane, which forms the pump volume for receiving and delivering the medical fluid and, in a state at least partially filled with the medical fluid, is elastically extended in such a way that a delivery pressure on the pump volume is produced. Accordingly, the pump units are each designed in the manner of an elastomer pump. Elastomer pumps are in principle known as such in the field of medical technology and can also be referred to as elastomer infusion pumps. The pump unit is preferably dimensioned in such a way that it is easily worn on a patient's body and can be used without an external energy supply, particularly in the context of outpatient infusion therapy. The energy for delivering the medical fluid is preferably provided alone by the elastically extended elastomer membrane.

In a further embodiment of the present disclosure, the first coupling element and the second coupling element are designed complementing each other in such a way that a fluid-conducting connection produced by means of the coupling elements is non-releasable. The coupling elements accordingly serve to allow the end user to connect the respective pump unit to the respective throttle unit once. After a fluid-conducting connection has been produced, it is no longer releasable without destruction. This embodiment of the present disclosure in particular counteracts unwanted release of the fluid-conducting connection. In this way, improved patient safety can be achieved. For this purpose, the coupling elements can be provided with complementary locking or snap-fit geometries which, in the connected state, prevent release.

In a further embodiment of the present disclosure, the throttle units each have a third coupling element, which is designed to connect the throttle unit, and thus the medical pump device, in a fluid-conducting manner to a patient-side access. The third coupling element is preferably arranged at the end of a hose line assigned to the throttle unit. The third coupling element is preferably in the form of a fluid connector known in principle in the field of medical technology.

For example, the third coupling element can be in the form of a Luer connector or an NRFit connector.

In a further embodiment of the present disclosure, the throttle units each have a filter element, which is provided for filtering the medical fluid. The filter element is arranged in a fluid-carrying cross section of the throttle unit. By means of the filter element a further improvement in patient safety can be achieved, since any particulate contaminants can be filtered out of the medical fluid as it is delivered through the throttle unit. Alternatively or in addition, the throttle units can each have an indicator element which is designed to display a flow state of a the medical fluid. For example, the indicator element can be designed in such a way that a flow and a non-flow of the fluid and thus two different flow states can be displayed.

In a further embodiment of the present disclosure, the pump units each have a filling state in which the respective pump volume is pre-filled with the medical fluid. Pre-filled means that the medical fluid has already been introduced into the pump volumes during the production of the kit. This embodiment of the present disclosure is particularly advantageous if the pump units are each designed in the manner of an elastomer pump and have an elastomer membrane which forms the pump volume. The pre-filled state of the pump units permits simplified handling of the kit, since the pump volume does not need to be filled subsequently by the user.

In a further embodiment of the present disclosure, the pump units each have a sealing element, by means of which the respective pump volume is openably sealed in a fluid-tight manner. The sealing element serves for fluid-tight closure of the pump volume pre-filled with the medical fluid. By means of this seal, it is possible to achieve improved hygiene and, ultimately, improved patient safety in the configuration of the medical pump device using the kit. The sealing element can in particular be in the form of a cap, a stopper, a screw closure or twist closure, a predetermined breaking element or the like. The seal can be produced by form-fit engagement, force-fit engagement and/or cohesive bonding.

In a further embodiment of the present disclosure, the pump units each have a pressure reducer, by means of which a delivery pressure of the medical fluid is reducible. The pressure reducer is connected in a fluid-conducting manner to the pump volume. Pressure reducers are in principle known as such in the field of flow technology. The pressure reducer can also be referred to as a pressure-reducing valve or reducer valve. The pressure reducer ensures that a pressure of the medical fluid at the outlet side of the pump unit does not exceed a defined limit value. The pressure reducer in particular counteracts a pressure-induced overdosage of the medical fluid and ultimately permits a further improvement in patient safety.

In a further embodiment of the present disclosure, a further group with a plurality of different functional units is provided, wherein the functional units have differently dimensioned properties, preferably in terms of flow mechanics, and wherein the functional units are each connectable in a fluid-conducting manner to one of the pump units and/or one of the throttle units. For the fluid-conducting connection to the pump units and/or the throttle units, the functional units can each be provided with corresponding coupling elements.

The present disclosure moreover relates to a kit for the modular configuration of a medical pump device for delivering a medical fluid, having at least one pump unit, which has a pump volume for receiving and delivering the medical fluid, wherein the pump unit has a first coupling element, which is designed in such a way that the pump volume is connectable in a fluid-conducting manner to a throttle unit, and having at least one throttle unit, which is designed to throttle the delivery of the medical fluid and for this purpose has a throttle effect, wherein the throttle unit has a second coupling element, which is designed in such a way that the throttle unit is connectable in a fluid-conducting manner to the pump volume of the pump unit, wherein the pump unit and the throttle unit are arranged spatially separated from each other. As regards the substantive and functional design of the at least one pump unit and of the at least one throttle unit, the statements made in connection with the above-described kit having a group consisting of a plurality of different pump units and a group consisting of a plurality of different throttle units apply correspondingly here. In order to avoid repetition, reference is expressly made to the relevant disclosure.

The present disclosure moreover relates to a medical pump device for delivering a medical fluid, wherein the medical pump device is characterized by a modular configuration using a kit according to the above description.

### BRIEF DESCRIPTION OF THE DRAWING FIGURES

Further advantages and features of the present disclosure are set out in the following description of a preferred illustrative embodiment, which is shown in the drawings.

FIG. 1 shows, in a highly simplified schematic view, an embodiment of a medical pump device according to the present disclosure, which is obtained in a modular configuration using an embodiment of a kit (FIG. 4) according to the present disclosure,

FIG. 2 shows, in a highly simplified schematic view, a pump unit and a throttle unit of the medical pump device according to FIG. 1 in an unconnected state,

FIG. 3 shows, in a further simplified schematic view, the medical pump device according to FIGS. 1 and 2, wherein further design features can be seen and the pump unit and the throttle unit are shown separate from each other,

FIG. 4 shows, in a highly simplified schematic block diagram, an embodiment of a kit according to the present disclosure, the medical pump device according to FIGS. 1 to 3 being configured using said kit,

FIG. 5 shows, in a view corresponding to FIG. 4, an embodiment of a kit according to the present disclosure with at least one pump unit and at least one throttle unit, and

FIG. 6 shows, in a highly simplified schematic block diagram, an embodiment variant of the kit according to FIG. 4, wherein the kit has a group with different functional units.

### DETAILED DESCRIPTION

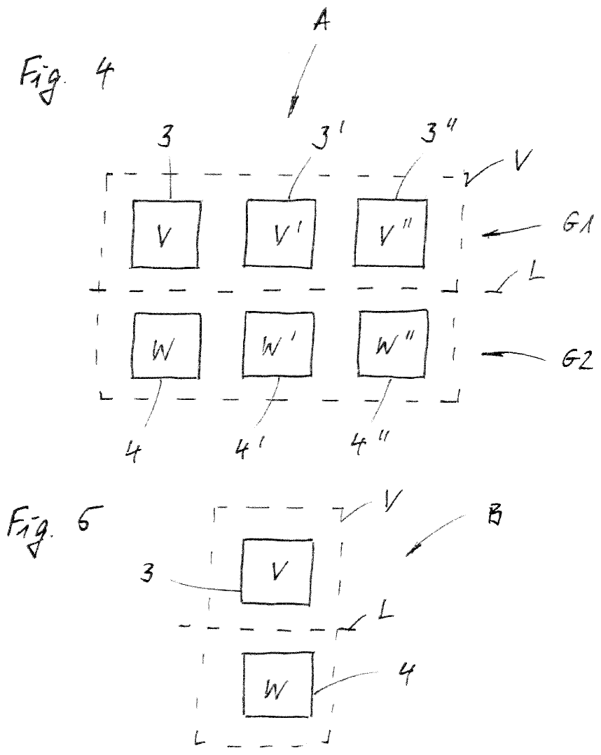

A medical pump device 1 according to FIGS. 1 to 3 is provided to deliver a medical fluid 2 in the context of outpatient and/or inpatient infusion therapy. In a manner described in more detail below, the medical pump device 1 is obtained in a modular configuration using a kit A according to FIG. 4. The substantive and functional design of the medical pump device 1 is first of all discussed in detail below.

The medical pump device 1 has a pump unit 3 and a throttle unit 4.

The pump unit 3 has a pump volume V, which is provided for receiving and delivering the medical fluid 2. The pump unit 3 is designed, in a manner described in more detail below, in such a way that the medical fluid 2 can be delivered by means of the pump unit 3 without an external and in particular electrical energy supply. The pump unit 3 moreover has a first coupling element 5. The first coupling element is designed in such a way that the pump volume V is connectable in a fluid-conducting manner to the throttle unit 4. In the present case, the first coupling element 5 is arranged at the end of a fluid line 6 which, at an end directed away from the first coupling element 5, is connected in a fluid-conducting manner to the pump volume V.

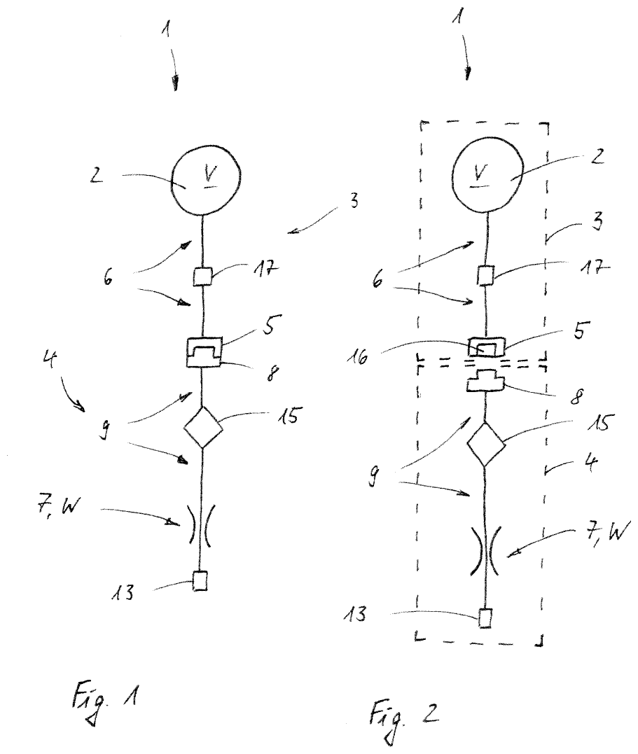

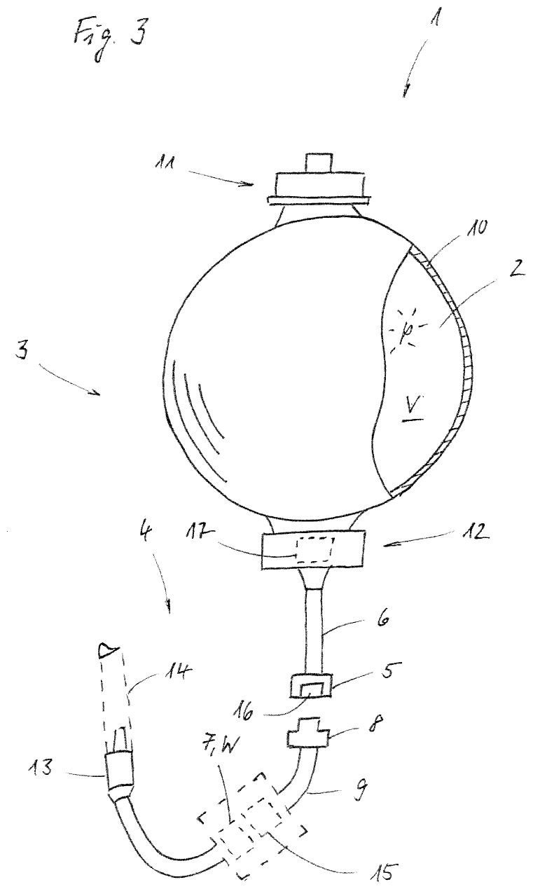

The throttle unit 4 is designed to throttle the delivery of the medical fluid 2 effected by means of the pump unit 3. For this purpose, the throttle unit 4 has a throttle element 7. The throttle element 7 is designed in such a way that the delivery of the medical fluid 2 is limited to a fixed flow rate. For this purpose, the throttle element 7 has an appropriate throttle effect W in terms of fluid technology. Moreover, the throttle unit 4 has a second coupling element 8, which is provided for fluid-conducting connection to the first coupling element 5 of the pump unit 3. For this purpose, the coupling elements 5, 8 are designed complementing each other. In the state shown in FIG. 1, the coupling elements 5, 8 are connected to each other in a fluid-conducting manner. In the state shown in FIG. 2, the coupling elements 5, 8 are by contrast separated from each other. The second coupling element 8 is arranged at the end of a fluid line 9 of the throttle unit 4. The fluid line 9 forms a fluid-conducting connection between the second coupling element 8 and the throttle element 7.

In the present case, the first coupling element 5 and the second coupling element 8 are designed complementing each other in such a way that a fluid-conducting connection effected by means of the coupling elements 5, 8, as shown in FIG. 1, is not releasable without destruction. For this purpose, the coupling elements 5, 8 can be provided with a correspondingly designed locking geometry and/or snap-fit geometry.

Further substantive and functional features of the medical pump device 1 will become clear from the detailed description of FIG. 3. It will be seen from the latter that the pump unit 3 has an elastomer membrane 10, which forms the pump volume V. The pump unit 3 has a filling state in which the pump volume V is filled with the medical fluid 2. The medical fluid 2 in the present case is a liquid medicament not described in any detail. In this filling state, the elastomer membrane 10 is flexibly extended like a balloon on account of a mechanical action of the medical fluid 2. In FIG. 3, the elastomer membrane 10 is illustrated with an exaggerated wall thickness for graphic reasons. By contrast, in a state when not filled with medical fluid 2, the elastomer membrane 10 is slack or at any rate elastically extended to a lesser extent. For the purpose of filling the pump volume V with the medical fluid 2, a reclosable filling nozzle 11 is provided here, which is attached in a fluid-tight manner to the elastomer membrane 10 in a way that is known in principle. The elastically extended elastomer membrane 10 produces a delivery pressure p on the pump volume V. By means of the delivery pressure p produced in this way, the medical fluid 2 is able to be delivered from the pump volume V into the fluid line 6 via an outlet nozzle 12, which is attached in a fluid-tight manner to the elastomer membrane 10 in a way that is known in principle. The fluid line 6 is attached in a fluid-conducting manner to the outlet nozzle 12 in a way that is known in principle. In the present case, the fluid line is in the form of a bendably flexible hose portion 6, wherein the first coupling element 5 is arranged at the end of the hose portion 6. In the present case, the pump unit 3 is designed in the manner of a medical elastomer pump. Pumps of this kind are basically known as such in the field of medical technology and can also be referred to as elastomer infusion pumps.

It will also be seen from FIG. 3 that the fluid line 9 is in the faun of a bendably flexible hose portion. The second coupling element 8 is arranged at the end of the hose portion 9 and, in the state shown in FIG. 3, is not connected to the first coupling element 5. At an end region of the hose portion 9 directed away from the second coupling element 8, a third coupling element 13 is arranged. The third coupling element 13 is designed for fluid-conducting connection of the throttle unit 4 and, when the coupling elements 5, 8 are connected to each other in a fluid-conducting manner, of the medical pump device 1 to a patient-side access (not shown more specifically in the drawings). In the present case, only a fluid line portion 14 assigned to the patient access is indicated by dashed lines and partially cut away. The third coupling element 13 is designed in the form of a Luer connector, as is known in principle in the field of medical technology. In an embodiment not shown in detail in the drawings, the third coupling element 13 can be in the faun of an NRFit connector. Further components of the throttle unit 4, in particular the throttle element 7, are illustrated only schematically and in a highly simplified manner in FIG. 3 and to this extent are indicated by dashed lines.

As will also be seen in particular from FIGS. 1 and 2, the throttle unit has a filter element 15. The filter element 15 is assigned to the fluid line 9 and, during delivery of the medical fluid 2, is flushed through by the latter and serves to filter any particulate contaminants out of the medical fluid 2.

Moreover, in the state when not connected to the throttle unit 4 as in FIGS. 2 and 3, the pump unit 3 has a sealing element 16. The sealing element 16 is assigned here to the first coupling element 5, although this does not necessarily have to be the case. The sealing element 16 serves to provide a fluid-tight seal of the pump volume V. Accordingly, in the state shown in FIGS. 2 and 3, the pump volume V is sealed openably in a fluid-tight manner by means of the sealing element 16. The sealing element 16 is designed here in the manner of a stopper, which is inserted in a fluid-tight manner at the front end into the first coupling element 5. In an embodiment not shown in detail in the drawings, the sealing element 16 can also be designed in such a way that the first coupling element 5 is openably sealed in a fluid-tight manner by the sealing element 16 by cohesive bonding. Before a manual connection of the coupling elements 5, 8, the sealing element 16 is manually removed and the pump volume V is thereby unsealed.

Moreover, the pump unit 3 has a pressure reducer 17. The pressure reducer 17 is assigned to the fluid line 6 and, during delivery of the medical fluid 2, is flushed through by the latter. Pressure reducers are fundamentally known as such in the field of fluid technology. The pressure reducer 17 is illustrated only schematically and in a highly simplified manner in the figures. FIG. 3 shows that the pressure reducer 17 is here integrated in the outlet nozzle 12. However, this is not necessarily the case. By means of the pressure reducer, the delivery pressure p can be reduced to a predefined and in particular constant pressure.

The medical pump device 1 is obtained in a modular configuration using the kit A shown in the block diagram in FIG. 4.

The kit A has a group G1 with a plurality of different pump units 3, 3′, 3″. The pump units 3, 3′, 3″ have differently dimensioned pump volumes V, V′, V″.

In the present case, the group G1 consists of three different pump units 3, 3′, 3″. However, the group G1 can also consist of only two different pump units or of considerably more than the illustrated three pump units 3, 3′, 3″. The pump volume V of the pump unit 3 measures 60 ml in the present case. The pump volume V′ of the pump unit 3′ measures 120 ml in the present case. The pump volume V″ of the pump unit 3″ measures 270 ml in the present case. In other words, the pump unit 3 is designed to administer 60 ml of the medical fluid 2, the pump unit 3′ is designed to administer 120 ml of the medical fluid 2, and the pump unit 3″ is designed to administer 270 ml of the medical fluid 2. Otherwise, as regards the substantive and functional design of the pump units 3′ and 3″, the disclosure concerning the pump unit 3, particularly in connection with FIGS. 1 to 3, correspondingly applies, and therefore reference is made to said disclosure in order to avoid repetition.

The kit A moreover has a group G2 with a plurality of different throttle units 4, 4′, 4″. The throttle units 4, 4′, 4″ have differently dimensioned throttle effects W, W′, W″. In the present case, a flow rate corresponding to the throttle effect W measures 5 ml/h, a flow rate corresponding to the throttle effect W′ measures 4 ml/h, and a flow rate corresponding to the throttle effect W″ measures 2 ml/h. Otherwise, as regards the substantive and functional design of the throttle units 4′ and 4″, the disclosure concerning the throttle unit 4 correspondingly applies, and therefore reference is made to said disclosure in order to avoid repetition. The group G2 in the present case comprises the three different throttle units 4, 4′, 4″. However, the group G2 can also consist of only two different throttle units or of considerably more than the illustrated three throttle units 4, 4′, 4″.

In an embodiment not illustrated, the throttle effects of the throttle units can be different in view of tolerance and/or accuracy of the achievable flow rates.

The pump units 3, 3′, 3″ and the throttle units 4, 4′, 4″ are arranged spatially separated from each other inside the kit A, as is indicated schematically by the dashed line L. Accordingly, the pump units 3, 3′, 3″ and the throttle units 4, 4′, 4″ are not connected to each other in a fluid-conducting manner inside the kit A. In the present case, the kit A has a packing arrangement V in which the groups G1, G2 are packed together. This does not rule out the possibility that the groups G1, G2 and/or the individual pump units 3, 3′, 3″ and the individual throttle units 4, 4′, 4″ are each additionally packed separately. The packing arrangement V is indicated only by dashed lines in FIG. 4.

The kit A serves for the modular configuration, by an end user, of the medical pump device 1 according to FIGS. 1 to 3 in particular. The present design of the kit A permits 9 different specifications as regards a combination of the pump volumes V, V′, V″ and of the throttle effects W, W′, W″, and also of the corresponding flow rates. Accordingly, the kit A allows an end user, for example a patient or medical personnel, to obtain a suitable modular configuration of a medical pump device adapted to the specific case of use. Depending on whether the case of use requires the administration of a smaller or larger quantity of the medical fluid 2 and a shorter or longer administration time, the end user can accordingly combine one of the pump units 3, 3′, 3″ with one of the throttle units 4, 4′, 4″ in an appropriate manner and connect them to each other by means of the coupling elements 5, 8 (cf. FIGS. 1 to 3). For example, the present combination of the pump volume V dimensioned to 60 ml and of the throttle effect W dimensioned to a flow rate of 5 ml/h permits an infusion time of 12 hours. However, if for example an infusion time of 15 hours or 30 hours is required and an administration of 60 ml of the medical fluid 2, the end user can combine the pump unit 3 with the throttle unit 4′ or the throttle unit 4″.

In the embodiment not illustrated, a selection in view of the accuracy of the throttle effect can be made to satisfy the individual application case. If an application case permits, for example, a comparatively great variation of the resulting infusion time, the final user can select a throttle unit with comparatively low accuracy of the achievable flow rate.

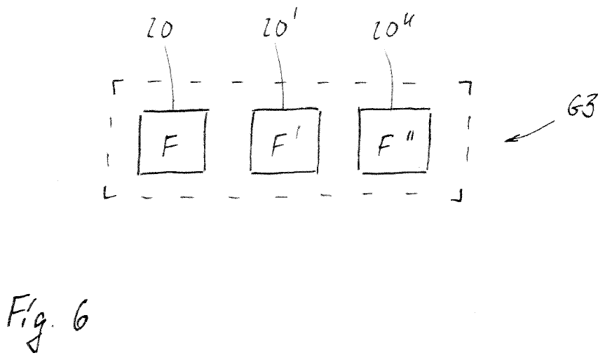

FIG. 5 shows a kit B. The medical pump device 1 according to FIGS. 1 to 3 can be obtained in a modular configuration using the kit B. For this purpose, the kit B has the pump unit 3 and the throttle unit 4. To avoid repetition, reference is made to the disclosure regarding kit A, which applies accordingly to kit B. Kit B differs from kit A only in that, instead of the groups G1, G2 with a plurality of pump units and throttle units, it includes only at least the one pump unit 3 and the at least one throttle unit 4.

FIG. 6 shows a group G3 with a plurality of different functional units 20, 20′, 20″. The different functional units 20, 20′, 20″ have differently dimensioned properties F, F′, F″ in terms of flow mechanics and are each connectable in a fluid-conducting manner to one of the pump units 3, 3′, 3″ and/or to one of the throttle units 4, 4′, 4″. Accordingly, to put it simply, the kit A shown in FIG. 4 can be extended by the further group G3.

1. A kit for the modular configuration of a medical pump device for delivering a medical fluid, the kit comprising:
a first group with a plurality of different pump units, which each have a pump volume for receiving and delivering the medical fluid,
wherein the pump units have differently dimensioned pump volumes,
and wherein the pump units each have a first coupling element, which is designed in such a way that the respective pump volume is connectable to a throttle unit in a fluid-conducting manner,
and a second group with a plurality of different throttle units, which are each designed to throttle the delivery of the medical fluid,
wherein the throttle units have differently dimensioned throttle effects,
wherein the throttle units each have a second coupling element, which is designed in such a way that the respective throttle unit is connectable in a fluid-conducting manner to a pump volume of one of the pump units,
wherein the pump units and the throttle units are arranged spatially separated from each other.

2. The kit according to claim 1, wherein the pump units each have an elastomer membrane, which forms the pump volume for receiving and delivering the medical fluid and, in a state at least partially filled with the medical fluid, is elastically extended in such a way that a delivery pressure on the pump volume is produced.

3. The kit according to claim 1, wherein the first coupling element and the second coupling element are designed complementing each other in such a way that a fluid-conducting connection produced by the coupling elements is non-releasable.

4. The kit according to claim 1, wherein the throttle units each have a third coupling element, which is designed to connect the throttle unit, and thus the medical pump device, in a fluid-conducting manner to a patient-side access.

5. The kit according to claim 1, wherein the throttle units each have a filter element, which is provided for filtering the medical fluid.

6. The kit according to claim 1, wherein the pump units each have a filling state in which the respective pump volume is pre-filled with the medical fluid.

7. The kit according to claim 6, wherein the pump units each have a sealing element, by which the respective pump volume is openably sealed in a fluid-tight manner.

8. The kit according to claim 1, wherein the pump units each have a pressure reducer, by means of which a delivery pressure of the medical fluid is reducible.

9. The kit according to claim 1, wherein a further group with a plurality of different functional units is provided, wherein the functional units have differently dimensioned properties, and wherein the functional units are each connectable in a fluid-conducting manner to one of the pump units and/or one of the throttle units.

10. A kit for the modular configuration of a medical pump device for delivering a medical fluid, the kit comprising:
at least one pump unit, which has a pump volume for receiving and delivering the medical fluid,
wherein the pump unit has a first coupling element, which is designed in such a way that the pump volume is connectable in a fluid-conducting manner to a throttle unit,
and at least one throttle unit, which is designed to throttle the delivery of the medical fluid and for this purpose has a throttle effect,
wherein the throttle unit has a second coupling element, which is designed in such a way that the throttle unit is connectable in a fluid-conducting manner to the pump volume of the pump unit,
wherein the pump unit and the throttle unit are arranged spatially separated from each other.

11. A medical pump device for delivering a medical fluid, the medical pump device having a modular configuration comprised of a kit according to claim 10.

12. A medical pump device for delivering a medical fluid, the medical pump device having a modular configuration comprised of a kit according to claim 1.
